(19) United States
(12) Patent Application Publication
(10) Pub. No.: US 2025/0230118 A1
(43) Pub. Date: Jul. 17, 2025

(54) TWO-STAGE PROCESS FOR PRODUCING TETRAALKYL 1,2,3,4 BUTANETETRACARBOXYLATES

(71) Applicant: Evonik Oxeno GmbH & Co. KG, Marl (DE)

(72) Inventors: Michael Grass, Haltern am See (DE); Johannes Kraft, Offenbach (DE); Michael Woelk-Fährmann, Marl (DE); Michael van Eickels, Recklinghausen (DE); Lena Bergup, Reken (DE); Stephan Becker, Marl (DE)

(73) Assignee: Evonik Oxeno GmbH & Co. KG, Marl (DE)

(21) Appl. No.: 19/010,391

(22) Filed: Jan. 6, 2025

(30) Foreign Application Priority Data
Jan. 11, 2024 (EP) 24151278.9

(51) Int. Cl. C07C 51/31; C07C 67/08

(52) U.S. Cl. CPC C07C 51/31; C07C 67/08

(57) ABSTRACT

A two-stage process produces tetraalkyl 1,2,3,4-butanetetracarboxylates having alkyl groups with 1 to 6 carbon atoms starting from tetrahydrophthalic anhydride (THPA). The process starts with an oxidation of tetrahydrophthalic anhydride (THPA), followed by a subsequent esterification of the resulting 1,2,3,4-butanetetracarboxylic acid with an alcohol having 1 to 6 carbon atoms.

### CROSS-REFERENCE TO RELATED APPLICATIONS

This patent application claims priority to European Patent Application No. 24151278.9, filed on Jan. 11, 2024, in the European Patent Office, the entire disclosure of which is hereby incorporated by reference herein.

### BACKGROUND OF THE INVENTION

### Field of the Invention

The present invention relates to a two-stage process for producing tetraalkyl 1,2,3,4-butanetetracarboxylates containing alkyl groups having 1 to 6 carbon atoms starting from tetrahydrophthalic anhydride (THPA). The process comprises the oxidation of tetrahydrophthalic anhydride (THPA) and the subsequent esterification of the resulting 1,2,3,4-butanetetracarboxylic acid with an alcohol having 1 to 6 carbon atoms.

### Description of Related Art

Tetraalkyl 1,2,3,4-butanetetracarboxylates are known in the chemical industry and have the following general structure

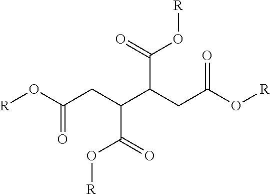

wherein all four radicals R represent an alkyl radical. These esters may be employed as plasticizers for plastics for example.

Tetraalkyl 1,2,3,4-butanetetracarboxylates may in principle be produced by chemical and electrochemical means. The chemical route proceeds via the synthesis of 1,2,3,4-butanetetracarboxylic acid and subsequent esterification with an alcohol to afford the corresponding tetraalkyl 1,2,3,4-butanetetracarboxylate. The electrochemical route proceeds via a hydrodimerization of dialkyl maleate occurring at the cathode. Such processes have in some cases already been described in the patent literature, for example in EP 0 816 533 A2, in WO 97/26389 A1 or in WO 02/42249 A1.

The known processes have the disadvantage that they either cannot be operated economically or cannot be operated sustainably on an industrial scale. An alternative route for producing the relevant tetraalkyl 1,2,3,4-butanetetracarboxylates is also to be provided.

### SUMMARY OF THE INVENTION

The process according to the invention is a process for producing tetraalkyl 1,2,3,4-butanetetracarboxylates comprising alkyl groups having 1 to 6 carbon atoms, wherein the process comprises the steps of:
a) oxidizing tetrahydrophthalic anhydride (THPA) in aqueous solution with hydrogen peroxide using a catalyst to produce a reaction solution containing at least 1,2,3,4-butanetetracarboxylic acid, unreacted or only partially reacted THPA and residual hydrogen peroxide,
b) separating the 1,2,3,4-butanetetracarboxylic acid from the reaction solution obtained in step (a);
c) esterifying the 1,2,3,4-butanetetracarboxylic acid with a C1- to C6-alcohol in the absence or presence of a catalyst;
d) separating the water formed in the esterification and the excess alcohol to obtain the tetraalkyl esters of 1,2,3,4-butanetetracarboxylic acid comprising alkyl groups having 1 to 6 carbon atoms.

The process according to the invention has the advantage that it employs well known and readily available raw materials such as tetrahydrophthalic anhydride (THPA) and hydrogen peroxide. Production of tetrahydrophthalic anhydride (THPA) is carried out by a Diels-Alder reaction of maleic anhydride with butadiene. The proposed process according to the present invention is therefore suitable for industrial scale production.

### DETAILED DESCRIPTION OF THE INVENTION

The first step a) of the process according to the invention is the oxidation of tetrahydrophthalic anhydride (THPA) with hydrogen peroxide. This oxidation is performed in aqueous solution using a catalyst to form a reaction solution containing at least butanetetracarboxylic acid, unconverted or only partially converted THPA and residual hydrogen peroxide. The reaction scheme for the aforementioned oxidation of THPA (1) to afford 1,2,3,4-butanetetracarboxylic acid (7) is shown below. The oxidation completes several intermediate steps to form at least the following intermediates:

1,2,3,6-Tetrahydrophthalic acid (2), 4,5-epoxycyclohexane-1,2-dicarboxylic acid (3), 4,5-dihydroxycyclohexane-1,2-dicarboxylic acid (4), 1,5-dipentanal-2,3-dicarboxylic acid (6) and 5-oxopentane-1,2,3-tricarboxylic acid (6).

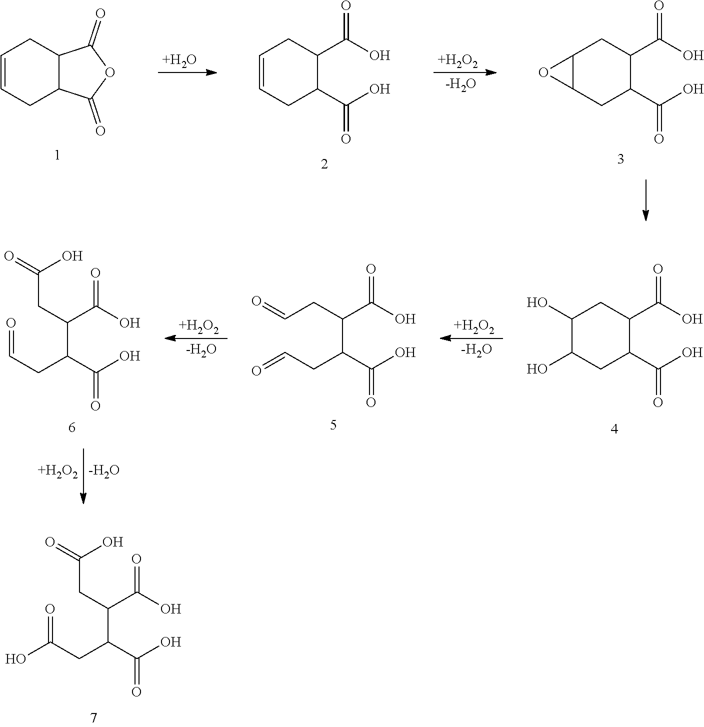

Tetrahydrophthalic anhydride (THPA) for use in the oxidation according to step a) is commercially available in purity levels of at least 99.5%. The hydrogen peroxide is preferably employed as an aqueous solution in the oxidation in step a) and it is particularly preferable when the hydrogen peroxide is added to the oxidation in step a) in the form of an at least 35% by weight hydrogen peroxide solution.

The hydrogen peroxide may in principle be employed in the oxidation in step a) in any desired amount so long as the reaction can proceed as desired. However, it is preferable according to the invention when the hydrogen peroxide, in particular the hydrogen peroxide solution, is added in an excess of 5% to 50%, preferably 15% to 40%, particularly preferably 20% to 30%.

The oxidation in step a) should preferably be carried out at elevated temperature to allow the reaction to proceed within an acceptable time. It is preferable when the reaction temperature in the oxidation in step a) is in the range between 50° C. and 100° C., preferably between 70° C. and 98° C., particularly preferably between 80° C. to 95° C. The pressure prevailing in the oxidation in step a) is less critical. It is preferable when the oxidation in step a) is performed at a pressure of 0.5 to 5 bar, preferably at ambient pressure.

The oxidation in step a) is still performed in the presence of a suitable catalyst. Catalysts which promote oxidation reactions are known to those skilled in the art. In the context of the present invention catalysts containing tungsten are preferred. Suitable examples of tungsten-containing catalysts include phosphotungstic acid and sodium tungstate, of which sodium tungstate is particularly preferably employed. It will be appreciated that the catalyst should be employed in a catalytically active amount. It is also possible to employ higher amounts of catalyst, even if this does not seem advantageous on cost grounds. In a preferred embodiment of the present invention the catalyst is employed in an amount of 0.01 to 0.1 mol/3 mol THPA in the oxidation in step a).

The oxidation in step a) may be carried out either in continuous mode or in batch mode. A person skilled in the art can select the mode according to the particular circumstances. The same applies to the plant engineering. The oxidation in step a) may be carried out in a single reactor but may also be carried out in a plurality of reactors connected in parallel or in series. Suitable reactors are generally known to those skilled in the art. One example of a suitable reactor for the oxidation in step a) is a continuous stirred tank reactor.

In terms of process engineering, attention may be paid to how the individual components are added in the oxidation. It is thus preferable according to the invention when the tetrahydrophthalic anhydride (THPA), the catalyst and water are initially charged in the reactor, the mixture is then heated and stirred, and hydrogen peroxide is added only subsequently to start the reaction.

In a further preferred embodiment of the present invention the reactor(s) are supplied with an inert gas during the oxidation in step a) of the process. This makes it possible to minimize the concentration of oxygen that may be formed through decomposition of hydrogen peroxide to avoid any problems that might arise due to the presence of oxygen. The inert gas employed may be any gas which is inert towards the oxidation in step a). The inert gas is preferably nitrogen, argon or helium. Nitrogen is particularly preferred. If an inert gas is employed the reaction solution formed in the oxidation is passed to a flash unit to degas the reactor outlet. This especially removes the inert gas.

The oxidation in step a) affords a reaction solution which contains at least 1,2,3,4-butanetetracarboxylic acid, unconverted or only partially converted THPA and residual hydrogen peroxide. The resulting butanetetracarboxylic acid is then separated from the reaction solution in the following step b), preferably by crystallization and subsequent filtration. The term “only partially converted THPA” refers to all intermediates formed in the reaction. This especially includes the intermediates (2) bis (6) recited in the reaction scheme shown above.

The separation of butanetetracarboxylic acid in step b) is preferably carried out by crystallization. A possible crystallization method is cooling crystallization where the reaction solution from step a) is transferred into a suitable crystallization apparatus and then the 1,2,3,4-butanetetracarboxylic acid is at least partially crystallized at a temperature in the range from 2° C. to 25° C., preferably 3° C. to 22° C. The cooling crystallization is preferably carried out at ambient pressure. As a result of the crystallization at least a portion of the 1,2,3,4-butanetetracarboxylic acid formed will crystallize out as solid.

Another option is evaporation crystallization where the reaction solution from step a) is transferred into a suitable crystallization apparatus and then the 1,2,3,4-butanetetracarboxylic acid is at least partially crystallized at a temperature in the range from 50° C. to 70° C., preferably 55° C. to 65° C. The reaction is preferably performed under vacuum, i.e. at a pressure which is lower than ambient pressure. As a result of the crystallization at least a portion of the 1,2,3,4-butanetetracarboxylic acid formed will crystallize out as solid.

In the evaporation crystallization the water present in the reaction solution will at least partially evaporate. In a particularly preferred embodiment of the present invention the condensation energy of the evaporating water can be utilized. The evaporated water is condensed in a heat exchanger and the energy used for the evaporation in the crystallization apparatus. In a preferred embodiment of the present invention the steam formed in the evaporation crystallization is used for thermal integration in such a way that initially a compression of the steam to a higher pressure level is carried out and subsequently energy from the compressed steam is transferred in one or more heat exchangers within the process, for example in the crystallization to heat the crystallization solution, to heat the inert gas or for heating during drying. This has the result that less external energy need be supplied.

Irrespective of which crystallization process is employed the 1,2,3,4-butanetetracarboxylic acid is present as a solid in the reaction solution or a part of the reaction solution. Separation from the solution is preferably performed using a filtration. Filtration processes, in some cases using a centrifuge, are familiar to those skilled in the art. After the filtration the 1,2,3,4-butanetetracarboxylic acid remains as a filter cake. The remaining reaction solution will be obtained as a liquid phase or a so-called mother liquor. The liquid phase/the mother liquor obtained after the filtration which contains at least unconverted or only partially converted THPA, the catalyst and residual hydrogen peroxide is returned to the oxidation in step a) optionally after further crystallization steps. The mother liquor may also contain the intermediates of the oxidation according to the reaction scheme shown above. If the intermediates are recycled to the oxidation, they can undergo further reaction to afford 1,2,3,4-butanetetracarboxylic acid. The recycling also allows the overall conversion to be increased.

The solid 1,2,3,4-butanetetracarboxylic acid obtained as a filter cake may be subjected to a washing step before use in the esterification in step c) to reduce the amount of residual catalyst or the amount of other impurities or to effect complete removal thereof. Washing of the solid 1,2,3,4-butanetetracarboxylic acid may be carried out using any suitable liquid, for example water or ethanol. It is particularly preferable to employ water for the washing, for example even the water that is evaporated in the evaporation crystallization and then recondensed.

Irrespective of whether the obtained, solid 1,2,3,4-butanetetracarboxylic acid has been washed, the esterification in step c) may be preceded by a drying. However, this drying is not mandatory. It is thus possible to conceive of an embodiment of the process according to the invention where after the separation in step b) the 1,2,3,4-butanetetracarboxylic acid is not dried before use in the esterification in step c).

However, it is also possible to conceive of an embodiment of the present process where after the separation in step b) the 1,2,3,4-butanetetracarboxylic acid is dried before use in the esterification in step c). Drying may be performed either at elevated temperatures or by freeze-drying.

However, a certain drying may also be carried out before the esterification in step c) when the esterification catalyst is added to the esterification in step c) only once the water content of the 1,2,3,4-butanetetracarboxylic acid has been reduced, preferably by heating to a temperature of not less than 70° C., preferably not less than 100° C.

The process thus comprises the following embodiments of the process where the reaction solution is subjected to a cooling crystallization and subsequently a filtration, wherein after the filtration a crystallized product (solid 1,2,3,4-butanetetracarboxylic acid) and a liquid mother liquor are formed, the liquid mother liquor being recycled to the reactor(s) in step a). The crystallized product is preferably washed with water and then dried.

In step c) of the process according to the invention the 1,2,3,4-butanetetracarboxylic acid produced in step a) and separated in step b) is subjected to an esterification with a C1- to C6-alcohol. This forms the desired product, i.e. tetraalkyl 1,2,3,4-butanetetracarboxylates comprising alkyl groups having 1 to 6 carbon atoms.

The C1- to C6-alcohol is preferably used in excess in the esterification in step c). In a preferred embodiment of the present invention the C₁ to C₆ alcohol is employed in an amount of 115% to 200%, particularly preferably in an amount of 120% to 150%, of the stoichiometric amount necessary for complete esterification.

The alcohol used for esterification in step c) is a C1- to C6-alcohol, in particular a C1- to C6-monoalcohol, i.e. an alcohol having only a single alcohol group. In a preferred embodiment of the present invention the esterification in step c) employs a C4-to C6-alcohol to form tetraalkyl 1,2,3,4-butanetetracarboxylates comprising alkyl groups having 4 to 6 carbon atoms.

Employable C4- to C6-alcohols include 1-butanol, 2-butanol, 2-methyl-1-propanol, 2-methyl-2-propanol, 1-pentanol, 2-pentanol, 3-pentanol, 2-methyl-1-butanol, 2-methyl-2-butanol, 3-methyl-1-butanol, 3-methyl-2-butanol, 2,2-dimethyl-1-propanol, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, 3-ethyl-1-butanol or mixtures of two or more thereof. The C4- to C6-alcohol employed is preferably 1-pentanol, 2-pentanol, 3-pentanol, 2-methyl-1-butanol, 2-methyl-2-butanol, 3-methyl-1-butanol, 3-methyl-2-butanol, 2,2-dimethyl-1-propanol or mixtures thereof. The C4-to C6-alcohol employed is particularly preferably a mixture of at least two from the group of 1-pentanol, 2-methyl-1-butanol and 3-methyl-1-butanol.

The esterification in step c) may be performed in the absence or in the presence of a catalyst. The esterification in step c) is preferably performed in the presence of a catalyst. Known catalyst systems suitable for esterification may in principle be used to this end. Suitable catalysts for the esterification to produce the C1- to C6-alkyl esters of 1,2,3,4-butanetetracarboxylic acid according to the invention are titanate catalysts, for example tetra-n-butyl titanate, zirconates or sulfonic acids.

The esterification in step c) to produce the esters according to the invention is preferably carried out at a temperature of 120° C. to 250° C., more preferably at a temperature of 140° C. to 230° C., particularly preferably at a temperature of 160° C. to 215° C. The pressure during the esterification should preferably not be excessively high since this would increase the boiling temperature and thus also the esterification temperature. The pressure during the esterification is thus in a range from 0.5 to 7 bar absolute, preferably 3 bar absolute or less, particularly preferably not less than 0.5 bar absolute. The esterification in step c) is very particularly preferably carried out at ambient pressure.

An esterification in step c) forms water as a result of the reaction of the acid group with the C1- to C6-alcohol. This water is also called water of reaction. In a preferred embodiment of the present invention at least a portion of the water of reaction formed is separated during the ongoing reaction. This can inter alia shift the equilibrium of the reaction in the right direction.

The progress of the esterification reaction can be monitored by monitoring a parameter. The acid number or the amount of water may be monitored for example. Monitoring by gas chromatography, where the proportion of reactants and/or products may be determined, is also possible. The reaction may also be monitored by online analysis.

Once the reaction has progressed to a sufficient extent the reaction may be terminated in a number of different ways. One option is initially destroying the catalyst by addition of an alkaline solution. This simultaneously saponifies any remaining acid. The reaction solution may then be worked up by known processes. To this end step d) comprises separating the water formed in the esterification and the excess alcohol to obtain the tetraalkyl esters of butanetetracarboxylic acid comprising alkyl groups having 1 to 6 carbon atoms. This separation is preferably carried out by thermal separation.

Another option for terminating the reaction is that of initially performing step d) which comprises separating the water formed in the esterification and the excess alcohol to obtain the tetraalkyl esters of butanetetracarboxylic acid comprising alkyl groups having 1 to 6 carbon atoms. This separation is preferably carried out by thermal separation. The catalyst is then destroyed by addition of an alkaline solution.

The C1- to C5-alkyl esters, preferably C4-to C6-alkyl esters, particularly preferably C5-alkyl esters, of 1,2,3,4-butanetetracarboxylic acid obtained according to the present invention have advantageous properties when used as plasticizers for polymers. The present invention thus further provides for the use of the C1- to C5-alkyl esters, preferably C4-to C6-alkyl esters, particularly preferably C5-alkyl esters, of 1,2,3,4-butanetetracarboxylic acid as a plasticizer for polymers. Suitable polymers are specified below, preference being given to PVC or vinyl chloride-containing copolymers.

The present invention further provides for a plasticizer composition which contains not only the C1- to C5-alkyl esters, preferably C4-to C6-alkyl esters, particularly preferably C5-alkyl esters, of 1,2,3,4-butanetetracarboxylic acid but also a further plasticizer. Depending on the intended application, the plasticizer composition may contain one or more additional plasticizers that are especially distinct from the inventive mixture of tetraisopentyl esters of 1,2,3,4-butanetetracarboxylic acid to specifically adjust the properties of the resulting plasticizer composition. According to a particularly preferred embodiment, the plasticizer composition comprises however less than 5% by weight, more preferably less than 0.5% by weight, particularly preferably less than 0.1% by weight, of phthalates.

The additional plasticizers in the inventive plasticizer composition may be selected from the group consisting of adipates, benzoates, for example monobenzoates or glycol dibenzoates, chlorinated hydrocarbons (so-called chloroparaffins), citrates, cyclohexanedicarboxylates, epoxidized fatty acid esters, epoxidized vegetable oils, epoxidized acylated glycerides, furandicarboxylates, phosphates, succinates, sulfonamides, sulfonates, terephthalates, isophthalates, trimellitates and oligomeric or polymeric esters based on adipic acid, succinic acid or sebacic acid. In a preferred embodiment of the present invention, the plasticizer composition comprises a further plasticizer selected from the group consisting of alkyl benzoates, alkylsulfonic esters of phenol, dialkyl adipates, glycerol esters, C4-C6-acids of polyols, trialkyl citrates, acetylated trialkyl citrates, glycol dibenzoates, trialkyl esters of trimellitic acid, dialkyl terephthalates, dialkyl phthalates, dialkyl isophthalates, esters of furandicarboxylic acid, dialkanoyl esters of dianhydrohexitols (for example isosorbide), epoxidized fatty acid alkyl esters, polymer plasticizers, for example polyadipates, and dialkyl esters of 1,2-, 1,3- or 1,4-cyclohexanedicarboxylic acid.

In a further preferred embodiment the further plasticizer present in the plasticizer composition is selected from the group consisting of C8-to C13-alkyl benzoates, C4-to C10-dialkyl adipates, pentaerythritol tetravalerate, acetylated trialkyl citrates having C4 to C9-alkyl groups, C4-to C10-trialkyl trimellitates, C4-to C9-dialkyl terephthalates, C4-to C13-dialkyl phthalates, especially C9-to C13-dialkyl phthalates and C4-to C10-dialkyl esters of 1,2-, 1,3- or 1,4-cyclohexanedicarboxylic acid.

The present invention thus also provides a plastics composition containing the inventive C1- to C5-alkyl esters, preferably C4-to C6-alkyl esters, particularly preferably C5-alkyl esters, of 1,2,3,4-butanetetracarboxylic acid or the plasticizer composition and one or more polymers.

Suitable polymers are preferably selected from the group consisting of PVC, homo- or copolymers based on ethylene, propylene, butadiene, vinyl acetate, glycidyl acrylate, glycidyl methacrylate, ethyl acrylate, butyl acrylate or methacrylate with alkoxy radicals of branched or unbranched alcohols having one to ten carbon atom(s), acrylonitrile or cyclic olefins, polyvinylidene chloride (PVDC), polyacrylates, in particular polymethyl methacrylate (PMMA), polyalkyl methacrylate (PAMA), polyureas, silylated polymers, fluoropolymers, especially polyvinylidene fluoride (PVDF), polytetrafluoroethylene (PTFE), polyvinyl acetate (PVAc), polyvinyl alcohol (PVA), polyvinyl acetals, especially polyvinyl butyral (PVB), polystyrene polymers, especially polystyrene (PS), expandable polystyrene (EPS), acrylonitrile-styrene-acrylate (ASA), styrene-acrylonitrile (SAN), acrylonitrile-butadiene-styrene (ABS), styrene-maleic anhydride copolymer (SMA), styrene-methacrylic acid copolymer, polyolefins, especially polyethylene (PE) or polypropylene (PP), thermoplastic polyolefins (TPO), polyethylene-vinyl acetate (EVA), polycarbonates, polyethylene terephthalate (PET), polybutylene terephthalate (PBT), polyoxymethylene (POM), polyamide (PA), polyethylene glycol (PEG), polyurethane (PU), thermoplastic polyurethane (TPU), polysulfides (PSu), biopolymers, especially polylactic acid (PLA), polyhydroxybutyral (PHB), polyhydroxyvaleric acid (PHV), polyesters, starch, cellulose and cellulose derivatives, especially nitrocellulose (NC), ethylcellulose (EC), cellulose acetate (CA), cellulose acetate/butyrate (CAB), rubber and silicones.

In a preferred embodiment at least one polymer or preferably at least 90% by weight of the two or more polymers in the plasticizer composition is/are selected from the group consisting of polyvinyl chloride (PVC), polyalkyl methacrylate (PAMA), polyvinyl butyral (PVB), polyurethane, polysulfides, polylactic acid (PLA), polyhydroxybutyral (PHB), nitrocellulose and copolymers of vinyl chloride with vinyl acetate or with butyl acrylate. PVC is particularly preferred among these.

The amount of the inventive mixture of tetraisopentyl esters of 1,2,3,4-butanetetracarboxylic acid or of the plasticizer composition in the plastics composition is preferably 5 to 150 parts by mass, preferably 10 to 120 parts by mass, particularly preferably 15 to 110 parts by mass and very particularly preferably 20 to 100 parts by mass per 100 parts by mass of polymer. However, compositions containing one or more polymers which comprise less than 20 parts by mass of the C1- to C5-alkyl esters, preferably C4-to C6-alkyl esters, particularly preferably C5-alkyl esters, of 1,2,3,4-butanetetracarboxylic acid per 100 parts by mass of polymer are also conceivable.

A further preferred subject of the present invention is a plastics composition containing the C1- to C5-alkyl esters, preferably C4-to C6-alkyl esters, particularly preferably C5-alkyl esters, of 1,2,3,4-butanetetracarboxylic acid and a rapidly gelating plasticizer selected from the group consisting of dibutyl terephthalate, di (iso) pentyl terephthalate, isodecyl benzoate, isononyl benzoate, acetyltributyl citrate, tributyl citrate, dipropylene glycol dibenzoate, diethylene glycol dibenzoate, triethylene glycol dibenzoate and mixtures of two or more thereof, and at least one polymer, preferably PVC.

The inventive plastics composition is preferably a constituent of an adhesive, of a sealing composition, of a coating composition, of a lacquer, of a paint, of a plastisol, of a dryblend, of a foam, of a synthetic leather, of a floor covering, particularly the topcoat or foam layer thereof, of a roofing membrane, of an underbody protection, of a fabric coating, of a cable, of a wire insulation, of a hose, of an extruded article, of a film, of an article in the field of automotive interiors, of a wallpaper, of an ink, of a toy, of a contact sheet, of a foodstuffs packaging or of a medical article, especially of a tube or of a blood bag.

A further subject of the present invention is therefore the use of the plastics composition in adhesives, sealing compositions, coating compositions, lacquers, paints, plastisols, foams, synthetic leathers, floor coverings, particularly the topcoat or foam layer, roofing membranes, underbody protection, fabric coatings, cables, wire insulation, hoses, extruded articles, films, in the field of automotive interiors, in wallpapers, inks, toys, contact sheets, foodstuffs packaging or medical articles, especially in tubes or blood bags.

The present invention is elucidated hereinbelow with reference to examples. The examples relate to preferred embodiments but are not to be understood as limiting the invention.

### EXAMPLES

### Production of 1,2,3,4-Butanetetracarboxylic Acid

Production of the 1,2,3,4-butanetetracarboxylic acid was performed in a glass stirred reactor with a heating jacket and an internal cooling coil. To this end tetrahydrophthalic anhydride (456 g or 3 mol) and sodium tungstate dihydrate (16.5 g or 0.05 mol) in water (1 L) were initially charged in the reactor, heated to 65° C. and stirred for about 45 min. Subsequently a 35% hydrogen peroxide solution (1457 g, 25% excess) is added over 40 minutes and the reaction commences. During the reaction the temperature is limited to not more than 90° C. by countercooling (water cooling via the internal cooling coil) and maintained until the desired conversion (about 6 hours).

The crystallization of the 1,2,3,4-butanetetracarboxylic acid from the reaction solution already occurs during cooling after termination. The reaction solution was then concentrated by evaporation of the water at 60° C. in a rotary evaporator. The crystallized 1,2,3,4-butanetetracarboxylic acid was separated by filtration and obtained as a white solid.

### Production of Tetraisopentyl Esters of 1,2,3,4-Butanetetracarboxylic Acid

An apparatus (three-necked flask, stirrer and cooler with water separator) was charged with the 1,2,3,4-butanetetracarboxylic acid as produced above and a 50:50 mixture of 2-methylbutanol and n-pentanol (25% alcohol excess) (2-methylbutanol: Sigma Aldrich, purity ≥99%; n-pentanol: Honeywell, purity ≥99%). Tetra-n-butyl titanate was added as catalyst (molar ratio of 1,2,3,4-butanetetracarboxylic acid:catalyst=500:1) and the reaction was started. The reaction was carried out with nitrogen sparging. The reactants were slowly heated to a reaction temperature of 200° C. Once the reaction temperature had been reached additional alcohol was metered in. It was ensured during metered addition that the reaction temperature did not fall below 200° C.

Water is continuously formed in the course of the esterification and forms an azeotrope with the alcohol. The azeotrope is condensed, and the water is then removed with a water separator and the alcohol returned to the reaction. Reaction progress was monitored at regular intervals via the AN until an AN of <0.5 mg of KOH per g sample was achieved. The excess alcohol is then distilled off at 160° C. under vacuum. After further cooling to 80° C. the remaining acid is neutralized by addition of alkaline solution and the catalyst destroyed. In the final step solids are separated from the product by filtration at 80° C.

1. A process for producing tetraalkyl 1,2,3,4-butanetetracarboxylates comprising alkyl groups having 1 to 6 carbon atoms, the process comprising:
a) oxidizing tetrahydrophthalic anhydride (THPA) in aqueous solution with hydrogen peroxide in presence of a catalyst to produce a reaction solution containing at least 1,2,3,4-butanetetracarboxylic acid, unreacted or only partially reacted THPA and residual hydrogen peroxide,
b) separating the 1,2,3,4-butanetetracarboxylic acid from the reaction solution obtained in (a);
c) esterifying the 1,2,3,4-butanetetracarboxylic acid with a C1- to C6-alcohol in absence or presence of a catalyst; and
d) separating water formed in the esterification and excess alcohol to obtain tetraalkyl esters of 1,2,3,4-butanetetracarboxylic acid comprising alkyl groups having 1 to 6 carbon atoms.

2. The process according to claim 1, wherein the esterification employs a C4-to C6-alcohol, thus forming tetraalkyl 1,2,3,4-butanetetracarboxylates comprising alkyl groups having 4 to 6 carbon atoms.

3. The process according to claim 2, wherein the employed C4-to C6-alcohol is 1-butanol, 2-butanol, 2-methyl-1-propanol, 2-methyl-2-propanol, 1-pentanol, 2-pentanol, 3-pentanol, 2-methyl-1-butanol, 2-methyl-2-butanol, 3-methyl-1-butanol, 3-methyl-2-butanol, 2,2-dimethyl-1-propanol, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, 3-ethyl-1-butanol or mixtures of two or more thereof.

4. The process according to claim 1, comprising:
adding hydrogen peroxide in an excess of 5% to 50%.

5. The process according to claim 1, wherein the catalyst employed in the oxidation in a) is a catalyst containing tungsten.

6. The process according to claim 1, comprising:
carrying out the separation of the 1,2,3,4-butanetetracarboxylic acid in b) by crystallization and subsequent filtration.

7. The process according to claim 6, wherein the 1,2,3,4-butanetetracarboxylic acid remains as a filter cake.

8. The process according to claim 6, comprising:
carrying out the crystallization of the 1,2,3,4-butanetetracarboxylic acid as a cooling crystallization or as an evaporation crystallization.

9. The process according to claim 8, comprising:
performing the cooling crystallization of the 1,2,3,4-butanetetracarboxylic acid at a temperature in a range from 2° C. to 25° C.

10. The process according to claim 8, comprising:
performing the evaporation crystallization of the 1,2,3,4-butanetetracarboxylic acid at a temperature in a range from 50° C. to 70° C., and optionally under vacuum.

11. The process according to claim 10, comprising:
subjecting a degassed reactor output to a cooling crystallization or evaporation crystallization and subsequently a filtration, wherein after the filtration a crystallized product and a liquid mother liquor are formed, the liquid mother liquor being recycled to the reactor.

12. The process according to claim 8, comprising:
employing a steam formed in the evaporation crystallization for thermal integration to initially carrying out a compression of the steam to a higher pressure level and subsequently transferring energy from the compressed steam in one or more heat exchangers within the process.

13. The process according to claim 1, comprising:
supplying an inert gas in the esterification in c).

14. The process according to claim 1, comprising:
drying, after the separation in b), the 1,2,3,4-butanetetracarboxylic acid before the esterification in c).

15. The process according to claim 1, comprising:
adding the esterification catalyst to the esterification in c) only once a water content of the 1,2,3,4-butanetetracarboxylic acid has been reduced.
